(19) United States
(12) Patent Application Publication
(10) Pub. No.: US 2007/0212336 A1
(43) Pub. Date: Sep. 13, 2007

(54) Methods for harvesting and storing autologous stem cells including blood derived hematopoietic stem cells and adipose derived mesenchymal stem cells

(76) Inventors: Loren Daniel Fulkerson, Yorba Linda, CA (US); James Drew DeOlden, Irvine, CA (US)

Correspondence Address: Donald Bollella, 21 Vetrina, Irvine, CA, 92606, US

(21) Appl. No.: 11/591,434

(22) Filed: Oct. 31, 2006

Related U.S. Application Data
(60) Provisional application No. 60/731,852, filed on Oct. 31, 2005.

(51) Int. Cl. A61K 35/12; A01N 1/02; C12N 5/08

(52) U.S. Cl. 424/93.7; 435/366; 435/2

(57) ABSTRACT

A method of collecting and storing stem cells from a patient is disclosed. The method includes the steps of obtaining a micro quantity of stem cells from the patient, cryogenically preserving the micro quantity of stem cells for a period of time, and reintroducing into the patient at least a portion of the micro quantity of stem cells by way of cellular expansion.

### CROSS-REFERENCE TO RELATED APPLICATIONS

This application claims the benefit of U.S. Provisional Application Ser. No. 60/731,852 filed Oct. 31, 2005, the entirety of which is herein incorporated by reference.

### BACKGROUND OF THE INVENTION

1. Field of the Invention

This invention relates in general to stem cells and, in particular, to the banking of stem cells. More specifically, but without restriction to the particular embodiments hereinafter described in accordance with the best mode of practice, this invention relates to methods for harvesting and storing autologous stem cells including blood derived hematopoietic stem cells and adipose derived mesenchymal stem cells.

2. General Discussion and Related Art

The market for stem cell technology, currently $500 million, has been independently estimated to grow to $30 billion by the year 2010. This will occur as new cellular therapeutics, based on adult stem cells, enter clinical practice and compete with, or complement, existing drug-based therapeutics.

Several firms are currently attempting to harvest and store stem cells from individuals on a pre-disease and prophylactic basis from one of two sources—mobilized peripheral blood via an apheresis process or adipose derived stem cells from fully invasive liposuction procedures. In both instances, the amount of stem cells collected is typically not in sufficient number to be of a therapeutic value by current medical standards. The necessary number of such cells is commonly referred to as the “Regenerative Therapeutic Amount”, or RTA. That standard is currently defined as the minimum number of stem cells required to reconstitute an entire immune system in a human by means of a bone marrow transplant. There is no universally accepted numerical consensus on this matter. The number of cells used for this procedure varies. Stated ranges are between 1×10⁶ per kg body weight to 10×10⁶ per kg body weight depending on the clinician's discretion. The RTA is generally achieved by several successive apheresis procedures prior to therapy. Conversely, the amount of stem cells required for specific tissue regeneration by most known standards is significantly less than the amounts stated above. Further, ongoing research and trials are suggestive that lower cell counts may be therapeutically useful.

At the present time, the firms offering elective collection of stem cells from either apheresed peripheral blood or adipocytes as the source of cells, collect at best enough cells to satisfy the lower range of the RTA for full body immune system reconstitution. The reason for this is the assumption that the effective RTA for newly emerging stem cell treatments is much less than that required to reconstitute an entire immune system.

It is understood by the inventors hereof that a stem cell service is essential for several reasons. Firstly, adult stem cell therapies are more likely to be developed before embryonic stems cell therapies due to significant governmental, legal, ethical and technical issues, which specifically affect embryonic stem cell therapeutics only. In addition, there is a greater acceptance of autologous stem cell transfer by both patients and regulatory authorities, and there currently exists a number of animal models that evidence the clear clinical potential of adult stem cell therapeutics. The inventors hereof believe that due to the current number of animal model trials, human trials are soon to follow and will use the knowledge gained in animal experiments. Other reasons why stem cell services will emmerge is that both private and public sectors have massively increased funding driven by perceived public demand, and it is understood that graft versus host disease is virtually non-existent with the use of autologous stem cells.

The art of stem cell research and related therapy has been contributed to by a number of those currently working in the art. For example, in the published application of Young et al., 2004/0033214, entitled “Pluripotent Embryonic-like Stem Cells, Compositions, Methods and Uses Thereof” there is disclosed a process for using a patients own stem cells during elective surgery. In one particular application discussed, a biopsy is removed from a patient wanting elective surgery. The stem cells are released from the biopsy by enzymatic digestion. After 30 days, the patient's autologous stem cells are transplanted back to the patient. In the published application to Hirose et al., 2004/0258673, entitled “Elective Collection and Banking of Autologous Peripheral Blood Stem Cells” there is disclosed a method of using one's own peripheral blood stem cells for future healthcare uses. The stem cells are collected while the patient is healthy or in a “pre-disease” state.

### OBJECTS AND SUMMARY OF THE INVENTION

It is, therefore, an object of the present invention to provide improved methods of collecting and storing stem cells.

The present invention is generally directed to refining existing collection processes that are gaining significant acceptance. The inventors hereof have provided a stem cell collection process and storage service that maintains adult stem cells for future use. The present methods and related processes have several advantages over the prior art. Upon banking one's stem cells, the participant will have immediately created a potential pre-disease source of stem cells so as to improve potential survival rates of certain types. of cancer based on existing therapeutic use of adult stem cells.

In addition, the patient will be more favorably placed to benefit from clinical advances in the future due to the expected increase in therapeutic applications of adult stem cells. These encompass a wide range of diseases and conditions, including heart disease, which is primarily associated with degenerative changes caused by aging. The principle applied here is that the earlier the stem cells are collected and stored, the less cellular degradation and the more useful the stem cells will be in the future.

By preserving a small supply of both blood derived and adipose derived stem cells on a pre-disease basis and making the cells available to the patient, the patient is ensuring that there are two different sources of stem cells from which to select for future cellular expansion. This is important as blood derived stem cells and adipose derived stem cells have different cell lineages and tend to expand to different cell types. This is beneficial as it allows the client-patient to select the best cell source to be expanded based on the clinical need at the time of the expansion process.

More specifically, these and other objects are attained in accordance with the present invention wherein there is provided a method of collecting and storing stem cells from a patient. This method includes the steps of obtaining a micro quantity of stem cells from the patient, cryogenically preserving the micro quantity of stem cells for a period of time, and reintroducing into the patient at least a portion of the micro quantity of stem cells by way of cellular expansion. This method may include collecting up to four units of non-mobilized peripheral blood from the patient. The collecting of the units of non-mobilized peripheral blood may be advantageously performed incrementally over a 12-month period. The collected units of non-mobilized peripheral blood are preferably tested for disease.

In one particular embodiment of the present invention, at least three units of non-mobilized peripheral blood are collected. this includes a first unit being collected at a first collection session, a second unit being collected at a second collection session, and a third unit being collected at a third collection session. During the third collection session, mesenchymal stem cells from the patient's adipose tissue may be collected. The mesenchymal stem cells from the patient's adipose tissue are preferably collected from the patient's abdominal region by a micro liposuction process or alternatively collected from other areas of the patient's body. In one specific embodiment hereof, the mesenchymal stem cells are collected from approximately 50 cc of adipose tissue. Once collected, the mesenchymal stem cells are preferably cryogenically preserved.

In accordance with one aspect of this invention, there is also provided the further step of fractionating the units of non-mobilized peripheral blood to isolate a white blood cell population from the peripheral blood. This is also provided the parallel step of fractionating the adipose tissue to isolate a mesenchymal stem cell population from the adipose tissue. According to this aspect of the present invention, there may be performed the step of cryogenically preserving the isolated white blood cell population, and cryogenically preserving the isolated mesenchymal stem cell population.

According to another aspect of the present invention there is further provided the step of obtaining a sample of plasma from the patient before the onset of any disease and storing the sample of plasma for later use. In accordance with this aspect, the sample of plasma is used as a fixed baseline reference in clinical diagnostic applications to allow a clinician to compare cells collected in a pre-disease state with those collected during a diseased state.

In accordance with yet another aspect of this invention there may be provided during the mesenchymal cell collection, an evaluation the patient's micro-vessel function to determine a measure of the client's vascular health.

### BRIEF DESCRIPTION OF THE DRAWING

Further objects of the present invention together with additional features contributing thereto and advantages accruing therefrom will be apparent from the following description of certain preferred embodiments of the invention which is shown in the accompanying drawing with like reference numerals indicating like components throughout, wherein:

FIG. 1 is a block diagram of the principal steps of a method according to the present invention; and

FIG. 2 is a block diagram illustrating further details of the present invention.

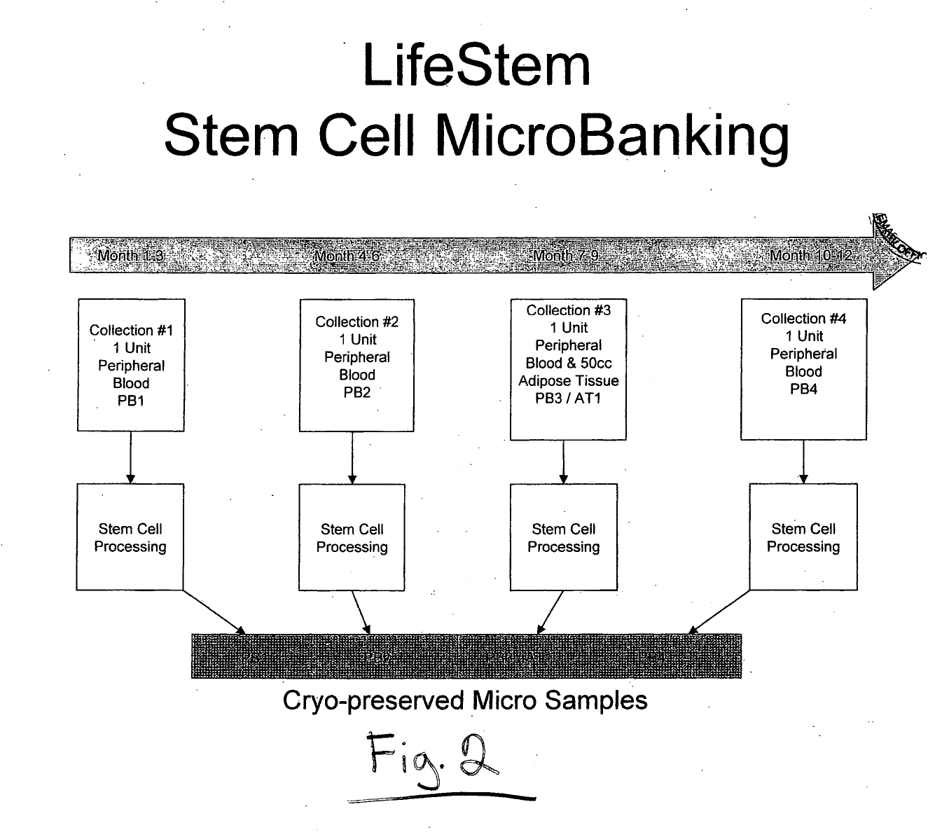

### DETAILED DESCRIPTION OF THE PREFERRED EMBODIMENTS

The present invention is based on the inventors' proposition that it is not necessary to harvest an RTA of stem cells at the time of collection, but rather that these cells can be collected in micro quantities and cryo-preserved for future cellular expansion at the time of reintroduction into the recipient. We further believe that it is in the recipient's best interest to store a small cell sample from two tissue sources—peripheral blood and adipose tissue. This concept is defined by the inventors hereof as “Stem Cell Microbanking”.

There are two principal reasons supporting the viability of the proposed method of collecting micro quantities of stem cells and cryo-preserving them for future cellular expansion at the time of reintroduction into the recipient. Firstly, animal studies and some clinical trials currently underway now are directly introducing stem cells locally into the dysfunctional tissue such as cardiac muscle. This generally reduces the number of cells required. Secondly, many researchers are developing techniques to enrich stem cells before delivery or enrich then expand the cells prior to delivery. The practical effect is again to greatly reduce the number of cells required in the starting population.

The micro-banking method of the present invention anticipates rapid future developments in these research areas, leading to FDA approved procedures, which can utilize a relatively small seed population of adult stem cells derived from blood and adipose tissue.

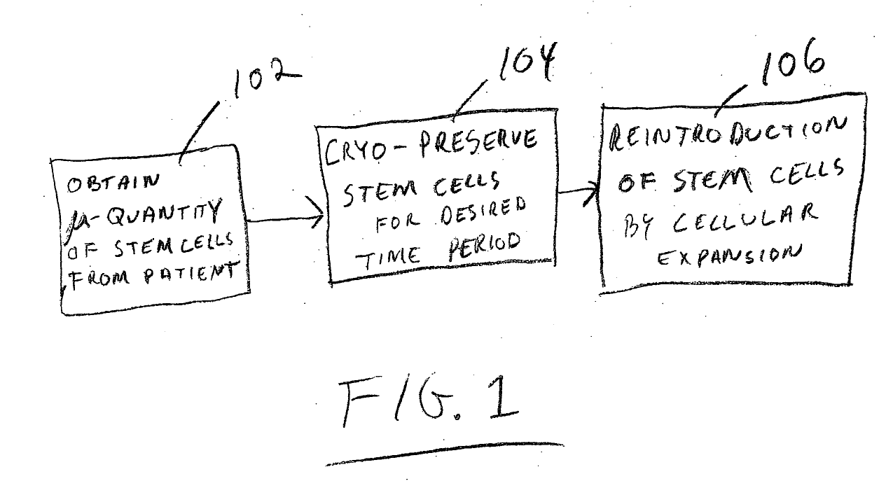

With reference now to FIG. 1, there is shown a block diagram of the principal steps of one method according to the present invention. At step 102, a micro quantity of stem cells is obtained from the patient. Next at step 104, the micro quantity of stem cells is preserved for a period of time. This preservation process is preferably performed cryogenically. And at step 106, at least a portion of the micro quantity of stem cells is reintroduced into the patient by way of cellular expansion.

More specifically, the present process involves the collection of up to 4 units of non-mobilized peripheral blood over a 12-month period by means that are standard to the medical industry and performed by a phlebotomist or another individual trained in peripheral blood collection. Each peripheral collection will be subjected to disease testing as is common with any blood donation process. During the third peripheral blood collection, the client will, through a micro liposuction process from the client's abdominal region or any other medically acceptable area, bank mesenchymal stem cells from approximately 50 cc of adipose tissue. The micro liposuction procedure will be performed by an M.D.

The units of non-mobilized peripheral blood and the adipose tissue will be fractionated according to industry standards to isolate the white blood cell population from the peripheral blood and the mesenchymal stem cell population from the adipose tissue. These two and separate refined stem cell populations will then be cryogenically preserved or “cryo-preserved” by means consistent with medical standards and stored together but in separate containers in such a manner that is consistent with current industry practices.

Additionally, a reference sample of plasma, or “cellular archive”, will be created which will allow the client to store a small population of blood plasma. This may have value as a fixed baseline reference in clinical diagnostic applications allowing the clinician to compare cells collected in a pre-disease state with those collected during the diseased state. The client will also be encouraged to receive a discount on service costs for the program by donating one unit of peripheral blood to be expanded immediately and used for research purposes.

The present method may also advantageously include, during the mesenchymal cell collection, a process whereby micro-vessel function will be evaluated, through a sample that is collected at the same time and through the same incision as the micro-liposuction. This provides a measure of the client's vascular health. When the micro-liposuction collection and 4^{th} unit of blood is given, the micro-collection process is complete and the harvested cells are cryo-preserved in the “Stem Cell Microbank” on behalf of the client.

While this invention has been described in detail with reference to certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

What is claimed is:

1. A method of collecting and storing stem cells from a patient, said method comprising the steps of:
obtaining a micro quantity of stem cells from the patient;
cryogenically preserving said micro quantity of stem cells for a period of time; and
reintroducing into the patient at least a portion of said micro quantity of stem cells by way of cellular expansion.

2. The method according to claim 1 wherein said step of obtaining a micro quantity of stem cells from the patient includes collecting up to four units of non-mobilized peripheral blood.

3. The method according to claim 2 wherein said collecting of said units of non-mobilized peripheral blood is performed incrementally over a 12-month period.

4. The method according to either claim 2 or 3 wherein each of said collected units of non-mobilized peripheral blood is tested for disease.

5. The method according to claim 2 wherein at least three units of non-mobilized peripheral blood are collected, a first unit being collected at a first collection session, a second unit being collected at a second collection session, and a third unit being collected at a third collection session.

6. The method according to claim 5 wherein during said third collection session, mesenchymal stem cells from the patient's adipose tissue are collected.

7. The method according to claim 6 wherein said mesenchymal stem cells from the patient's adipose tissue are collected from the patient's abdominal region by a micro liposuction process.

8. The method according to either claim 6 or 7 wherein said mesenchymal stem cells are collected from approximately 50 cc of adipose tissue.

9. The method according to any one of claims 6, 7, or 8 wherein said mesenchymal stem cells are cryogenically preserved.

10. The method according to any one of claims 2, 3, 4, 5, 6, 7, or 8 including the further step of fractionating said units of non-mobilized peripheral blood to isolate a white blood cell population from the peripheral blood.

11. The method according to any one of claims 6, 7, or 8 including the further step of fractionating said adipose tissue to isolate a mesenchymal stem cell population from said adipose tissue.

12. The method of claim 10 further including the step of cryogenically preserving said isolated white blood cell population.

13. The method of claim 11 further including the step of cryogenically preserving said isolated niesenchymal stem cell population.

14. The method according to any one of the above claims 1 to 13 including the further step of obtaining a sample of plasma from the patient before the onset of any disease and storing said sample of plasma for later use.

15. The method according to claim 14 wherein said sample of plasma is used as a fixed baseline reference in clinical diagnostic applications to allow a clinician to compare cells collected in a pre-disease state with those collected during a diseased state.

16. The method according to any one of claims 6, 7, 8, or 10 wherein during the mesenchymal cell collection, the patient's micro-vessel function is evaluated to determine a measure of the client's vascular health.
